(19) United States
(12) Patent Application Publication
(10) Pub. No.: US 2008/0161928 A1
(43) Pub. Date: Jul. 3, 2008

(54) COMPLIANT INTERVERTEBRAL PROSTHETIC DEVICES WITH MOTION CONSTRAINING TETHERS

(75) Inventor: Hai H. TRIEU, Cordova, TN (US)

Correspondence Address: HESLIN ROTHENBERG FARLEY & MESITI P.C., 5 COLUMBIA CIRCLE, ALBANY, NY, 12203, US

(73) Assignee: WARSAW ORTHOPEDIC, INC., Warsaw, IN (US)

(21) Appl. No.: 11/616,388

(22) Filed: Dec. 27, 2006

(51) Int. Cl. A61F 2/44; A61B 17/08

(52) U.S. Cl. 623/17.16; 623/17.11; 606/151

(57) ABSTRACT

An intervertebral prosthetic device is provided for implanting within an intervertebral disc space between first and second vertebral bodies. The device includes first and second components respectively adapted to engage the first and second vertebral bodies. A non-articular, elongate flexible core component is interposed between the first and second components, and one or more flat tethers are coupled to the first and second components to bind together the components and the core to constrain motion of the intervertebral prosthetic device when in operable position between the vertebral bodies. In various embodiments, the first and second components include first and second covers, and the elongate, flexible core includes regions of different elasticity. Further, the non-articular core is fixedly secured to the first and second components, and the flat tether is a flexible, braided textile-based structure. The prosthetic device is a posteriorally inserted intervertebral prosthetic device in various configurations.

### CROSS-REFERENCE TO RELATED APPLICATIONS

This application contains subject matter which is related to the subject matter of the following applications, which are hereby incorporated herein by reference in their entirety:

“Hybrid Intervertebral Disc System”, Hai. Trieu, U.S. Ser. No. 10/765,260, filed Jan. 27, 2004, and published on Jul. 28, 2005 as U.S. Patent Application Publication No. U.S. 2005/0165485 A1;

“Intervertebral Prosthetic Disc”, Heinz et al., U.S. Ser. No. 11/343,935, filed Jan. 31, 2006; and

“Posterior Articular Disc and Method for Implantation”, Allard et al., U.S. Ser. No. 11/460,887, filed Jul. 28, 2006.

### TECHNICAL FIELD

The present invention relates generally to spinal implants and methods, and more particularly, to intervertebral prosthetic joint devices and methods for use in total or partial replacement of a natural intervertebral disc.

### BACKGROUND OF THE INVENTION

In the treatment of disease, injuries and malformations affecting spinal motion segments, and especially those affecting disc tissue, it has been known to remove some or all of a degenerated, ruptured or otherwise failing disc. In cases involving intervertebral disc tissue that has been removed, or is otherwise absent from a spinal motion segment, corrective measures are typically desirable.

In one approach, adjacent vertebrae are fused together using transplanted bone tissue, an artificial fusion component, or other compositions or devices. Spinal fusion procedures, however, have raised concerns in the medical community that the biomechanical rigidity of the intervertebral fusion may predispose neighboring spinal motion segments to rapid deterioration. Unlike a natural intervertebral disc, spinal fusion prevents the fused vertebrae from pivoting and rotating with respect to one another. Such lack of mobility tends to increase stress on adjacent spinal motion segments. Additionally, conditions may develop within adjacent spinal motion segments, including disc degeneration, disc herniation, instability, spinal stenosis, spondylosis and facet joint arthritis as a result of the spinal fusion. Consequently, many patients may require additional disc removal and/or another type of surgical procedure as a result of the spinal fusion. Alternatives to spinal fusion are therefore desirable.

Alternative approaches to bone grafting employ a manufactured implant made of a synthetic material that is biologically compatible with a body in the vertebrae. There have been extensive attempts at developing acceptable prosthetic implants that can be used to replace an intervertebral disc and yet maintain the stability and range of motion of the intervertebral disc space between adjacent vertebrae. While many types of prosthetic devices have been proposed, there remains a need in the art for further enhanced intervertebral prosthetic disc devices and methods of implanting thereof.

### SUMMARY OF THE INVENTION

The shortcomings of the prior art are overcome and additional advantages are provided, in one aspect, through provision of an intervertebral prosthetic device which includes a first component and a second component. The first component is configured to engage a first vertebral body and the second component is configured to engage a second vertebral body. A non-articular, elongate flexible core component is interposed between and fixedly secured to the first and second components to bias the first and second components in spaced relation. At least one flat tether connects the first component and the second component to further bind together the first component, the non-articular, elongate flexible core component, and the second component, and to constrain motion of the intervertebral prosthetic device when in operable position within an intervertebral disc space between the first and second vertebral bodies, and subject to at least one of a flexion or extension force, a lateral bending force or an axial rotation force.

In another aspect, an intervertebral prosthetic device is provided which includes a first component adapted to engage a first vertebral body, and a second component adapted to engage a second vertebral body. The device further includes a non-articular, elongate flexible core component interposed between and secured to the first and second components. The non-articular, elongate flexible core component is coupled to the first component and to the second component, and biases the first and second components in spaced relation. Further, the non-articular, elongate flexible core component includes regions of different elasticity. The device further includes at least one flat tether connecting the first component and the second component to further bind together the first component, the non-articular, elongate flexible core component and the second component, and to constrain motion of the intervertebral prosthetic device when in an operable position within an intervertebral disc space between the first and second vertebral bodies, and subject to at least one of a flexion or extension force, a lateral bending force or an axial rotation force.

In a further aspect, an intervertebral prosthetic device is provided which includes a first component adapted to engage a first vertebral body, and a second component adapted to engage a second vertebral body, as well as a non-articular, elongate flexible core component disposed between and secured to the first and second components. The prosthetic device further includes at least one tether connecting the first component and the second component to further bind together the first component, the non-articular, elongate flexible core component and the second component, and to constrain motion of the intervertebral prosthetic device when posteriorally inserted in an operable position within an intervertebral disc space between the first and second vertebral bodies, and subject to at least one of a flexion or extension force, a lateral bending force or an axial rotation force. Additionally, the first component and the second component each have a length that extends upon cortical bone of opposing sides of an apophyseal ring of the corresponding vertebral body of the first and second vertebral bodies, and a width that is smaller than the length, and wherein the non-articular, flexible core component has a length and width at most equal to a length and width, respectively, of the first component and the second component.

In a yet further aspect, a method for implanting an intervertebral prosthetic device into an intervertebral disc space is provided. The method includes: obtaining an intervertebral prosthetic device including first and second components adapted to respectively engage first and second vertebral bodies, a non-articular, elongate flexible core component interposed between and fixedly secured to the first and second components to bias the first and second components in spaced relation, and at least one flat tether connecting the first and second components to further bind together and constrain motion of the intervertebral prosthetic device; surgically accessing an intervertebral disc space through an opening on a lateral side of the intervertebral disc space; and inserting the intervertebral prosthetic device into the intervertebral disc space through the opening on the lateral side of the intervertebral disc space and positioning the first component in engagement with the first vertebral body and the second component in engagement with the second vertebral body.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 depicts a lateral view of a portion of a human vertebral column;

FIG. 2 depicts a lateral view of a pair of adjacent vertebra of a vertebral column;

FIG. 3 is a top sectional plan view of a vertebra;

FIG. 4 is a lateral view of a portion of a vertebral column posteriorally receiving an intervertebral implant, in accordance with an aspect of the present invention;

FIG. 5 is a top sectional view of an intervertebral disc space with bilateral, posteriorally-inserted intervertebral prosthetic devices implanted therein, in accordance with an aspect of the present invention;

FIG. 6 is a lateral sectional view of an intervertebral disc space and intervertebral prosthetic device implanted therein, in accordance with an aspect of the present invention;

FIG. 7 is a perspective view of one embodiment of an intervertebral prosthetic device, in accordance with an aspect of the present invention;

FIG. 7A is a cross-sectional elevational view of the intervertebral prosthetic device of FIG. 7, taken along line 7A-7A, in accordance with an aspect of the present invention; and

FIG. 7B is a cross-sectional elevational view of an alternate embodiment of an intervertebral prosthetic device, in accordance with an aspect of the present invention;

FIGS. 8A-8F depict alternate embodiments of cover configurations, or alternatively, of endplate configurations employable in an intervertebral prosthetic device, in accordance with an aspect of the present invention;

FIGS. 9A-9F depict alternate embodiments of flat tether configurations for connecting the first component, non-articular, elongate flexible core component and second component of an intervertebral prosthetic device, in accordance with an aspect of the present invention;

FIGS. 10A-10L depict alternate embodiments of core component configurations for an intervertebral prosthetic device, in accordance with an aspect of the present invention;

FIG. 11 is a top sectional view of an intervertebral disc space and two intervertebral prosthetic devices illustrating a bilateral posterior implant process, in accordance with an aspect of the present invention;

FIG. 12 is a top sectional view of an intervertebral disc space illustrating an alternate unilateral posterior process for implanting an intervertebral prosthetic device, in accordance with an aspect of the present invention;

FIG. 13 is a top sectional view of an intervertebral disc space illustrating a posterior process for bilaterally implanting kidney-shaped intervertebral prosthetic devices, in accordance with an aspect of the present invention;

FIG. 14 is a top sectional view of an intervertebral disc space illustrating a posterior process for bilaterally implanting semi-circular intervertebral prosthetic devices, in accordance with an aspect of the present invention;

FIG. 15 is a top sectional view of an intervertebral disc space illustrating a posterior process for bilaterally implanting triangular-shaped intervertebral prosthetic devices, in accordance with an aspect of the present invention;

FIG. 16 is a top sectional view of an intervertebral disc space illustrating a posterior process for bilaterally implanting trapezoidal-shaped intervertebral prosthetic devices, in accordance with an aspect of the present invention;

FIG. 17 is a top sectional view of an intervertebral disc space illustrating a posterior process for bilaterally implanting mating rectangular-shaped intervertebral prosthetic devices, in accordance with an aspect of the present invention;

FIG. 18 is a top sectional view of an intervertebral disc space illustrating a posterior process for bilaterally implanting mating semi-toroidal-shaped intervertebral prosthetic devices, in accordance with an aspect of the present invention;

FIG. 19 is a top sectional view of an intervertebral disc space illustrating an alternative posterior process for bilaterally implanting differently-sized, rectangular-shaped intervertebral prosthetic devices, in accordance with an aspect of the present invention;

FIG. 20 is a top sectional view of an intervertebral disc space illustrating an alternative posterior process for bilaterally implanting differently-sized, kidney-shaped intervertebral prosthetic devices, in accordance with an aspect of the present invention;

FIG. 21 is a top sectional view of an intervertebral disc space during a posterior process for implanting two intervertebral prosthetic devices, in accordance with an aspect of the present invention;

FIG. 22 is a top sectional view of the intervertebral disc space of FIG. 21 at a further step in the posterior process for implanting two intervertebral prosthetic devices, in accordance with an aspect of the present invention;

FIG. 23 is a top sectional view of the intervertebral disc space of FIG. 22, at another step in the posterior process for implanting two intervertebral prosthetic devices, in accordance with an aspect of the present invention;

FIG. 24 is top sectional view of the intervertebral disc space of FIG. 23, at another step in the posterior process for implanting two intervertebral prosthetic devices, in accordance with an aspect of the present invention;

FIG. 25 is a top sectional view of an intervertebral disc space illustrating a step in an alternate posterior process for implanting intervertebral prosthetic devices, in accordance with an aspect of the present invention; and

FIG. 26 is a top sectional view of the intervertebral disc space of FIG. 25, at another step in the alternate posterior process for implanting intervertebral prosthetic devices, in accordance with an aspect of the present invention.

### BEST MODE FOR CARRYING OUT THE INVENTION

The present invention relates generally to vertebral reconstructive devices, and more particularly, to a functional intervertebral prosthetic disc device and related methods of implantation. For purposes of promoting an understanding of the principles of the invention, reference is made below to the embodiments, or examples, illustrated in the drawings and specific language is used to describe the same. It will nevertheless be understood that no limitation on the scope of the invention is thereby intended. Any alternations and further modifications in the described embodiments, and any further applications of the principles of the invention as described herein are contemplated as would normally occur to one skilled in the art to which the invention relates.

In human anatomy, the spine is a generally flexible column that can take tensile and compressive loads. The spine also allows bending motion and provides a place of attachment for tendons, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs absorb the compressive and tensile loads to which the spinal column may be subjected. At the same time, the intervertebral discs allow adjacent vertebral bodies to move relative to each other a limited amount, particularly during bending, or flexure, of the spine. Thus, the intervertebral discs are under constant muscular and/or gravitational pressure and generally are the first parts of the lumbar spine to show signs of deterioration.

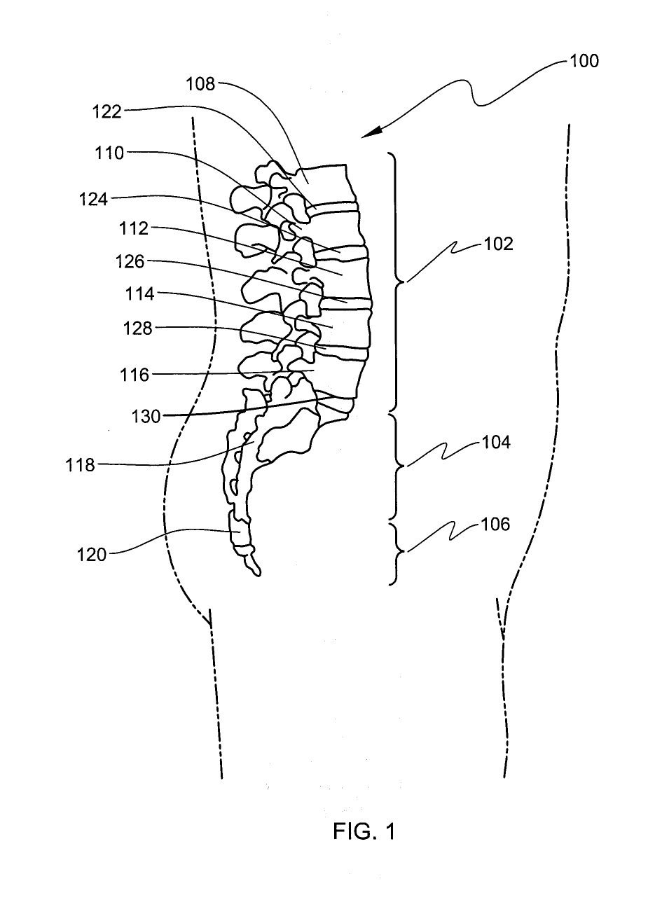

Referring now to the figures, and initially to FIG. 1, a portion of a vertebral column 100 is shown. As depicted, vertebral column 100 includes a lumbar region 102, a sacral region 104, and a coccygeal region 106. As is known in the art, vertebral column 100 also includes a cervical region and a thoracic region. For clarity and ease of discussion, the cervical region and the thoracic region are not illustrated.

As shown in FIG. 1, lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As also depicted in FIG. 1, a first intervertebral lumbar disc 122 is disposed between first lumbar vertebra 108 and second lumbar vertebra 110. A second intervertebral lumbar disc 124 is disposed between second lumbar vertebra 110 and third lumbar vertebra 112. A third intervertebral lumbar disc 126 is disposed between third lumbar vertebra 112 and fourth lumbar vertebra 114. Further, a fourth intervertebral lumbar disc 128 is disposed between fourth lumbar vertebra 114 and fifth lumbar vertebra 116. Additionally, a fifth intervertebral lumbar disc 130 is disposed between fifth lumbar vertebra 116 and sacrum 118.

In one particular embodiment, if one of the intervertebral lumbar discs 122, 124, 126, 128, 130 is diseased, degenerated, damaged, or otherwise in need of replacement, that intervertebral lumbar disc can be at least partially removed and replaced with an intervertebral prosthetic disc according to one or more of the embodiments described herein. In one embodiment, a portion of the intervertebral lumbar disc 122, 124, 126, 128, 130 is removed via a discectomy, or similar surgical procedure, well known in the art. Further, removal of intervertebral lumbar disc material can result in the formation of an intervertebral disc space (not shown) between two adjacent lumbar vertebrae.

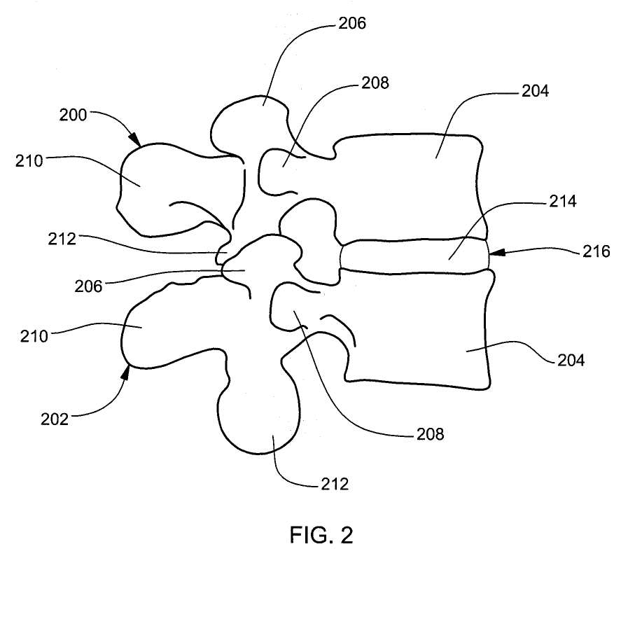

FIG. 2 depicts a detailed lateral view of two adjacent vertebra, e.g., two of the lumbar vertebra 108, 110, 112, 113, 116 shown in FIG. 1. In particular, FIG. 2 illustrates a superior vertebra 200 and an inferior vertebra 202. Each vertebra 200, 202 includes a vertebral body 204, a superior articular process 206, a transverse process 208, a spinous process 210 and an inferior articular process 212. FIG. 2 further illustrates that an intervertebral disc space 214 can be established between superior vertebra 200 and inferior vertebra 202 by removing the intervertebral disc 216. As described in greater detail below, an intervertebral prosthetic device, according to one or more of the embodiments described herein, can be inserted into intervertebral disc space 214 between superior vertebra 200 and inferior vertebra 202.

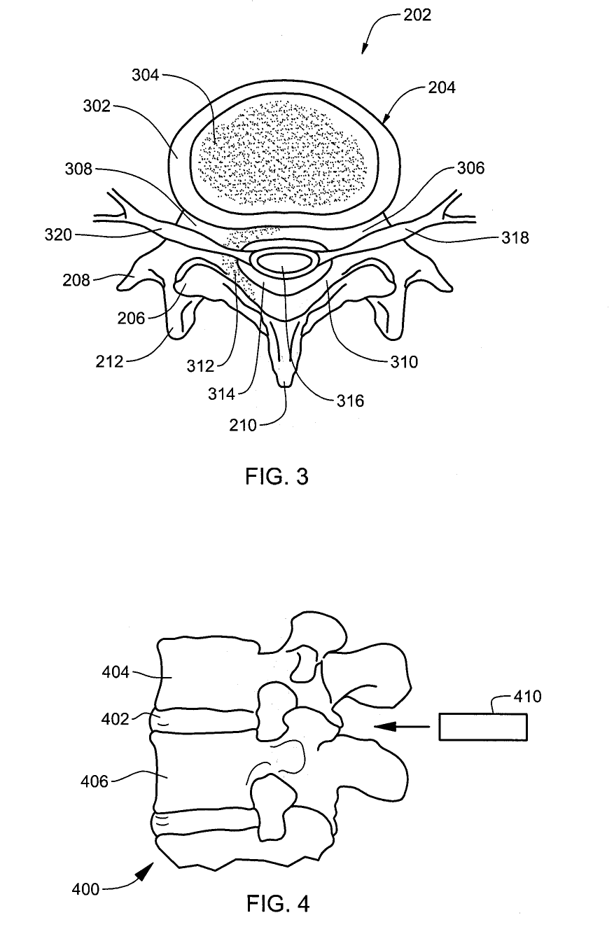

Referring to FIG. 3, a vertebra, e.g., inferior vertebra 202 (of FIG. 2), is illustrated in top plan view. As shown, vertebral body 204 of inferior vertebra 202 includes a cortical rim 302 composed of cortical bone. Also, vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, i.e., those structures described above in conjunction with FIGS. 2 & 3. The first and second cervical vertebrae are structurally different than the rest of the vertebrae in order to support the skull.

FIG. 4 illustrates a vertebral joint 400 which includes an intervertebral disc 402 extending between vertebrae 404, 406. Disc 402 may be partially or entirely removed and an intervertebral implant 410 inserted between the vertebrae 404, 406 to preserve motion within joint 400. Although the illustration of FIG. 4 generally depicts vertebral joint 400 as a lumbar vertebral joint, it should be understood that the devices and methods of this disclosure are applicable to all regions of a vertebral column, including the cervical and thoracic regions. Additionally, although the illustration of FIG. 4 generally depicts a posterior approach for insertion of implant 410, other approaches, such as a lateral or anterior approach, may alternatively be employed.

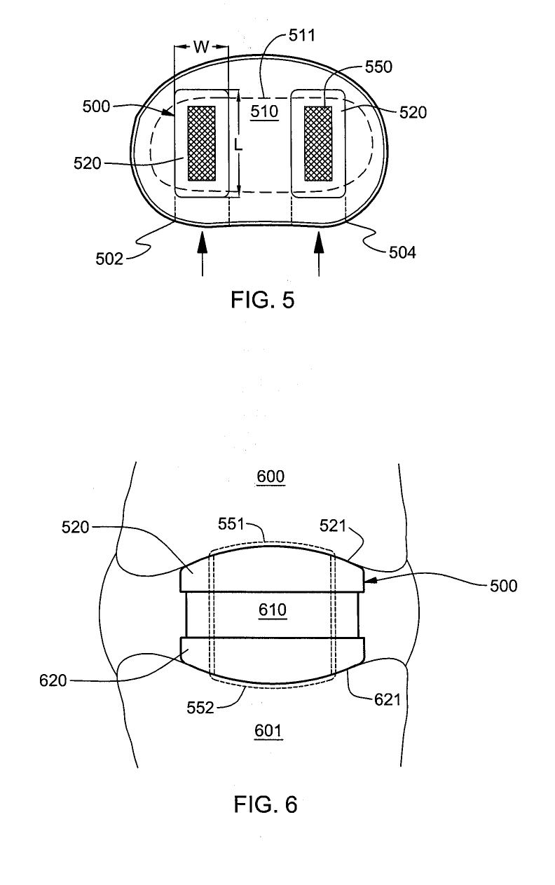

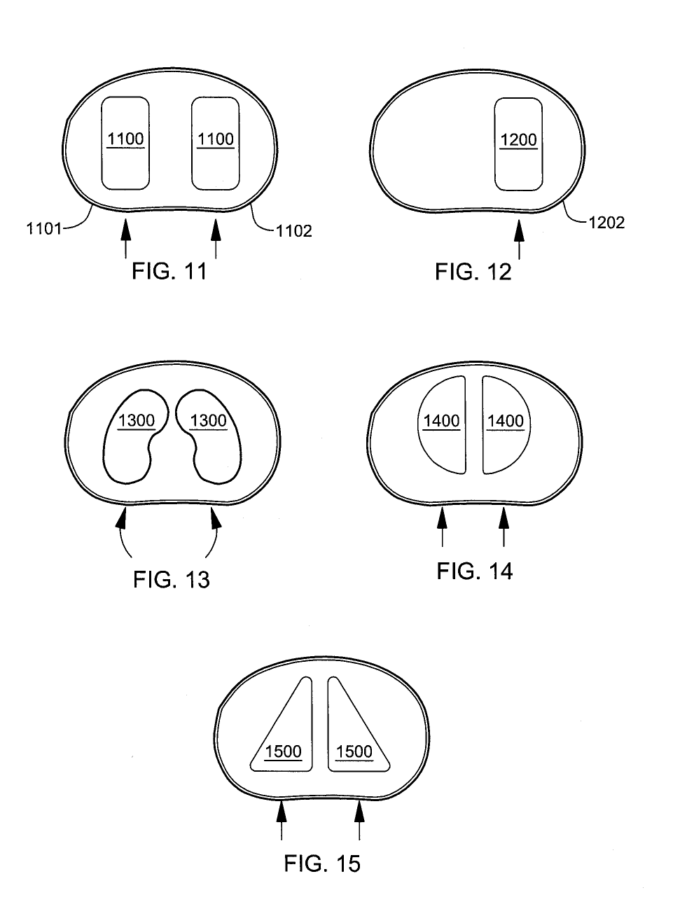

FIGS. 5-10L detail various intervertebral prosthetic device configurations, in accordance with aspects of the present invention. FIGS. 11-27 further illustrate various device configurations and depict bilateral and unilateral posterior implant processes which could be employed, in accordance with certain aspects of the present invention.

Referring first to FIGS. 5-7A, one embodiment of an intervertebral prosthetic device 500 is shown. Further, FIG. 5 illustrates a bilateral approach for posteriorally implanting two intervertebral disc devices 500 into an intervertebral disc space 510. In this embodiment, intervertebral prosthetic devices 500 are rectangular-shaped, however, as described further below, other elongate shapes such as kidney, oval, oblong, semi-circular, semi-toroidal, trapezoidal, triangular, etc., are also contemplated. The implant process includes (in one embodiment) creating an incision in the patient's back and forming a posterior unilateral opening on each lateral side 502, 504 of the intervertebral disc space. The opening may be any size required to accept a single intervertebral prosthetic device configured as described herein. For example, an 11 mm opening may be suitable. Through this opening, instrumentation may be inserted to evacuate remaining disc tissue. Instrumentation may also be inserted to mill or otherwise dislocate bone to fashion a path, track or recess in one or both of the vertebral endplates adjacent to the intervertebral disc space. It is understood that in certain embodiments, no bone removal may be needed. The disc space may be extracted through the milling procedure and/or subsequent insertion procedures.

In one aspect, an intervertebral prosthetic device in accordance with the present invention includes first and second components adapted to respectively engage adjacent first and second vertebral bodies of the intervertebral disc space. FIG. 5 illustrates a superior, first component 520, while FIG. 6 depicts first component 520 and an inferior, second component 620. These components 520, 620 each have a length L (FIG. 5) that extends upon cortical bone of opposing sides of an apophyseal ring 511 of the corresponding vertebral body of the first and second vertebral bodies defining the intervertebral disc space 510 within which the intervertebral prosthetic device 500 is implanted. The components further have a width W that is smaller than this length L. By way of specific example, components 520, 620 may each have a length L in the range of 18-30 mm, and a width W less than 15 mm. Additionally, the overall height of the intervertebral prosthetic device 500 may be in the range of 8-18 mm.

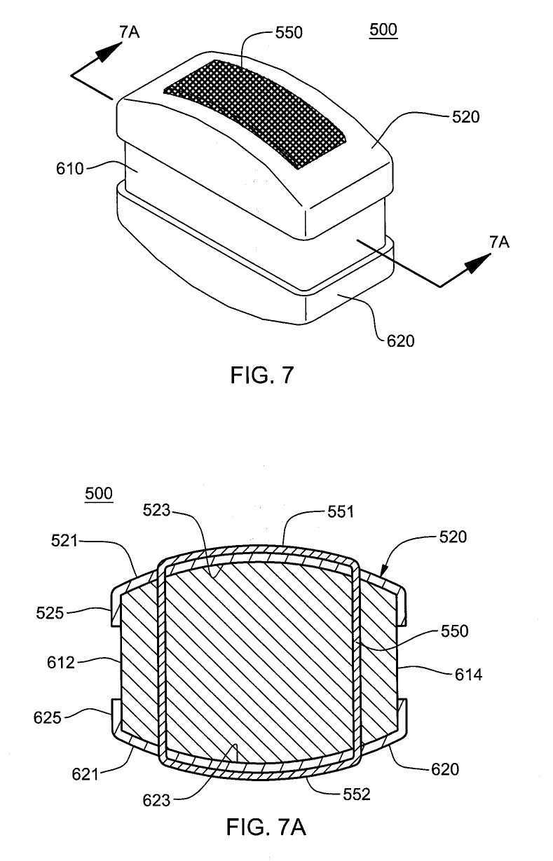

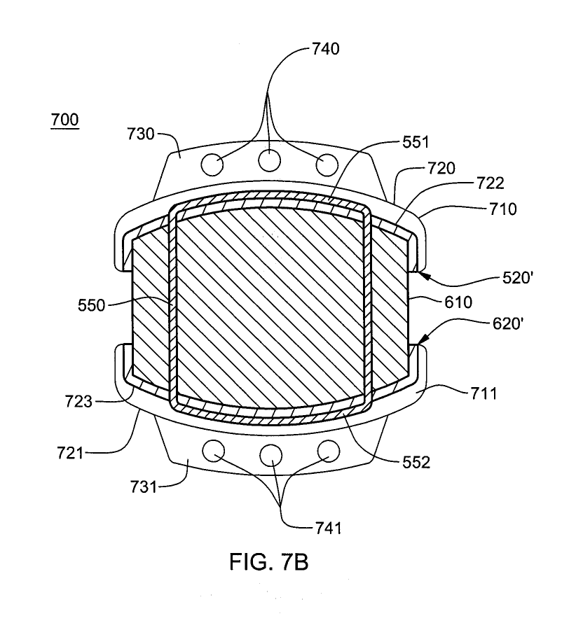

As best shown in FIGS. 6, 7 & 7A, first and second components 520, 620 of intervertebral prosthetic device 500 are (in this embodiment) endplates of the prosthetic device, with an elongate flexible core component 610 being disposed therebetween. Each endplate 520, 620 includes an exterior surface 521, 621 and an interior surface 523, 623. In this embodiment, exterior surfaces 521, 621 are convex to mate with a concavity in a respective vertebral endplate of the adjacent vertebral bodies when inserted within an intervertebral disc space. Further, interior surfaces 523, 623 are concave and configured to receive in mating engagement a respective convex surface of elongate flexible core component 610. In alternate embodiments, interior surfaces 523, 623 of endplates 520, 620 may be flat and smooth, with the corresponding mating surfaces of elongate flexible core component 610 also being flat. Further, each endplate in this example is configured with a circumferential lip 525, 625 sized to matably receive the elongate flexible core component in fixed position between the endplates. In all embodiments, however, the interfaces between first component 520 and elongate flexible core component 610, as well as between second component 620 and elongate flexible core 610 are non-articulating with respect to each other. Thus, in the embodiments described herein, the flexible core component is referred to below as a non-articular, elongate flexible core component.

Endplates 520, 620 may be formed of any suitable biocompatible material including metals such as cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

The non-articular, elongate flexible core component 610 includes a flexible body which, in one embodiment, is a unitary core component, as illustrated in FIG. 7A. In the embodiment illustrated, first and second end surfaces 612, 614 of non-articular, elongate flexible core component 610 are relatively flat and parallel. If desired, non-articular, elongate flexible core component 610 may be adhesively attached to first component 520 and to second component 620 to ensure that the core component is non-articulating relative to these components. Other coupling mechanisms (not shown) such as ridges and grooves may alternatively or additionally be employed to ensure non-articular securement of the elongate flexible core component to both of the first and second components. In further alternative embodiments, the non-articular, elongate flexible core component may have one or more curved end surfaces or have end surfaces angled with respect to one another.

Non-articular, elongate flexible core component 610 may be formed from one or more resilient materials which have a lower modulus than the endplate materials. Suitable flexible core materials may include polymeric elastomers such as polyolefin rubbers; polyurethanes (including polyetherurethane, polycarbonate urethane, and polyurethane with or without surface modified endgroups); copolymers of silicone and polyurethane with or without surface modified endgroups; silicones; and hydrogels. Polyisobutylene rubber, polyisoprene rubber, neoprene rubber, nitrile rubber, and/or vulcanized rubber of 5-methyl-1,4-hexadiene may also be suitable.

As shown in FIGS. 5-7A, intervertebral prosthetic device 500 further includes a flat tether 550, which in one embodiment, is a flexible, braided textile-based structure. The tether is flat in that the width of the tether is greater than the thickness of the tether. Further, the flat tether is preferably oriented such that the width of the tether is disposed transverse to the length dimension of the elongate core component. (In certain other embodiments described herein below, the width of the flat tether may be disposed to extend parallel to the length dimension of the elongate core component.) By way of specific example, tether 550 might be an elastic, woven textile material approximately 1-3 mm thick and 5-10 mm wide.

As shown in FIG. 7A, tether 550 extends through first component 520 and through second component 620 to form a loop. Appropriately sized elongate openings (not shown) are provided in first and second components 520, 620 to allow for passage of the looped, flat tether therethrough. These openings are then sealed to prevent wear debris from traveling inward into contact with the flexible core component. The looped tether further extends through appropriate openings or channels formed in non-articular, elongate flexible core component 610. One or multiple loops may be employed to bind together first component 520, non-articular, elongate flexible core component 610 and second component 620.

In operation, flat tether 550 flexes yet constrains motion of the intervertebral prosthetic device when in an operable position between a first and a second vertebral bodies 600, 601 (FIG. 6). Advantageously, flat tether 550 when oriented as depicted in the figures, provides maximum stability and strength when the device is inserted in an intervertebral disc space, as illustrated in FIG. 5, and subject to a flexion or extension force. Additionally, when disposed in an intervertebral disc space between the fourth lumbar vertebra and the fifth lumbar vertebra, or between the fifth lumbar vertebra and the sacrum, flat tether 550 tenses and functions to reinforce the intervertebral prosthetic device when a shear load is applied to the device due to the angle of the intervertebral disc space.

As an enhancement, the openings in the first and second components 520, 620, as well as the openings or channels formed in non-articular, elongate flexible core component 610, may be modified, treated, coated or lined to enhance the wear resistance and articulating properties of the flat tether relative to the first and second components, as well as relative to the flexible core component. These wear resistant and articulation properties may be provided by cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may be suitable. Polymer materials may also be used including any member of the PAEK family such as PEEK, carbon-reinforced PAEK, or PEKK; polysulfone, polyetherimide; polyimide; UHMWPE; and/or cross-linked UHMWPE. Polyolefin rubbers, polyurethanes, copolymers of silicone and polyurethane, and hydrogels may also provide wear resistance and articulation properties. Wear resistant characteristics may also or alternatively be provided by modifications such as cross-linking and metal ion implantation.

As shown in FIGS. 6 & 7A, flat tether 550 includes a first portion 551 exposed at exterior surface 521 of first component 520 and a second portion 552 exposed at exterior surface 621 of second component 620. Thus, first portion 551 and second portion 552 of tether 550 physically contact the respective vertebral endplates of the adjacent vertebral bodies 600, 601 when intervertebral prosthetic device 500 is implanted within the intervertebral disc space between the adjacent vertebrae. These exposed portions 551, 552 of tether 550 may be coated with a biocompatible and osteoconductive material such as hydroxyapatite (HA), tricalcium phosphate (TCP), and/or calcium carbonate to promote bone in-growth and fixation. Alternatively, osteoinductive coatings, such as proteins from transforming growth factor (TGF), beta superfamily, or bone-morphogenic proteins, such as BMP2 or BMP7, may be used. Further, exterior surfaces 521, 621 of the first and second components 520, 620 may also include features or coatings (not shown) which enhance fixation of the implanted prosthesis. For example, these surfaces may be roughened, such as by chemical etching, bead-blasting, sanding, grinding, serrating and/or diamond cutting. Further, these roughened surfaces may also be coated with a biocompatible material to promote bone in-growth and fixation.

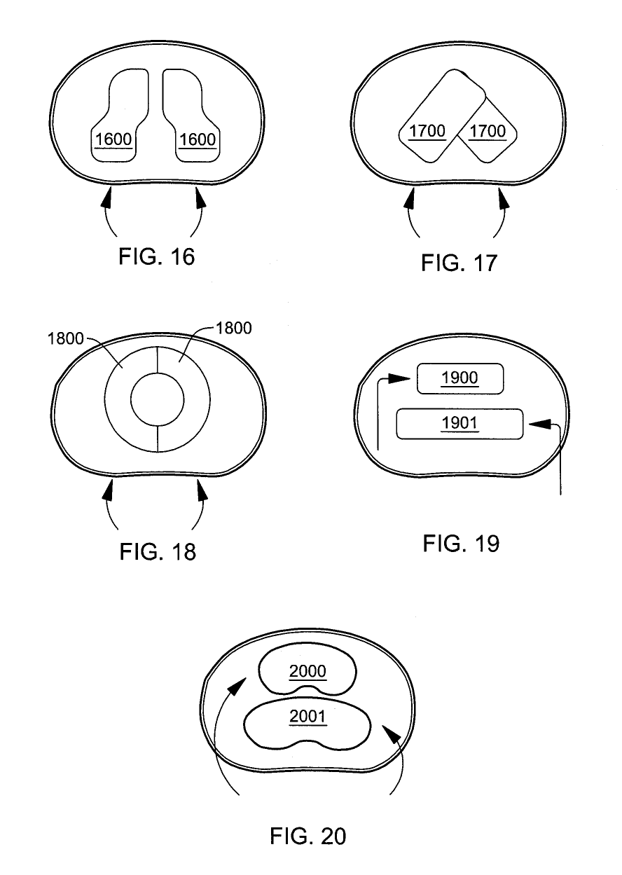

Although the embodiment of FIGS. 5-7A depicts a rectangular prosthetic device, other configurations may be employed. FIG. 13 depicts kidney-shaped devices, FIG. 14, semi-circular devices, FIG. 15, triangular-shaped devices, FIG. 16, trapezoidal-shaped devices, FIG. 17, rectangular mating-shaped endplates, and FIG. 18, mating semi-toroidal-shaped devices. Other shapes will also be apparent to those skilled in the art. The cross-sectional top viewed geometry of the non-articular, elongate flexible core component would be similarly shaped to that of the first and second endplates and be sized to ensure securement of the core component to the endplates, for example, via frictional or slight compressive coupling of the core component into the respective endplates. More particularly, the length and width of the elongate flexible core component is at most equal to the corresponding length L and width W (see FIG. 5) of the first and second endplates. In the embodiment of FIGS. 5-7A, the elongate flexible core is shown to have a slightly smaller length and width than the endplate structures due to the need to fit within the inwardly projecting circumferential lips 525, 625 of the endplates 520, 620.

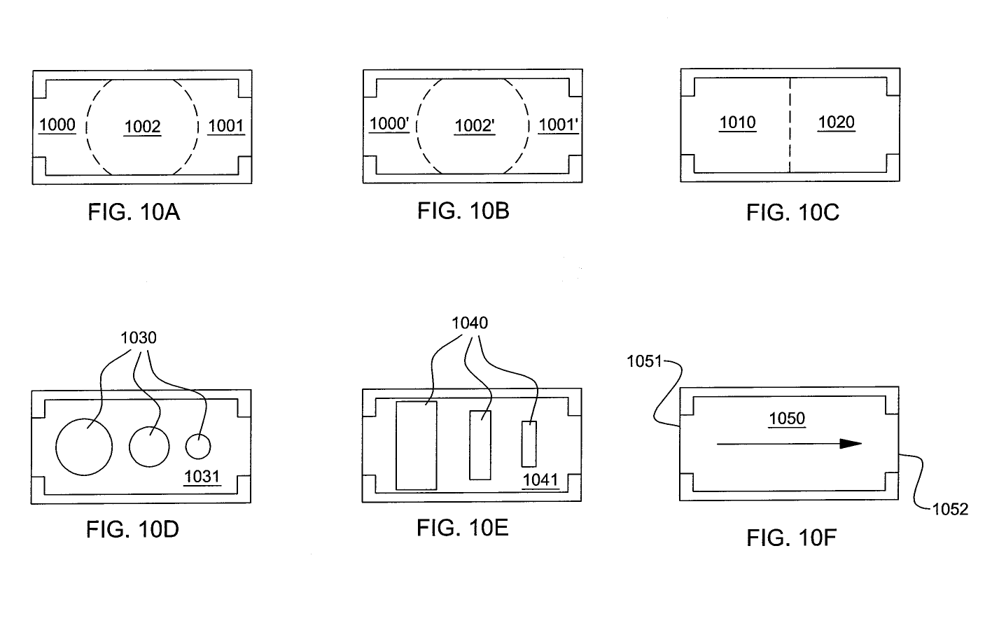

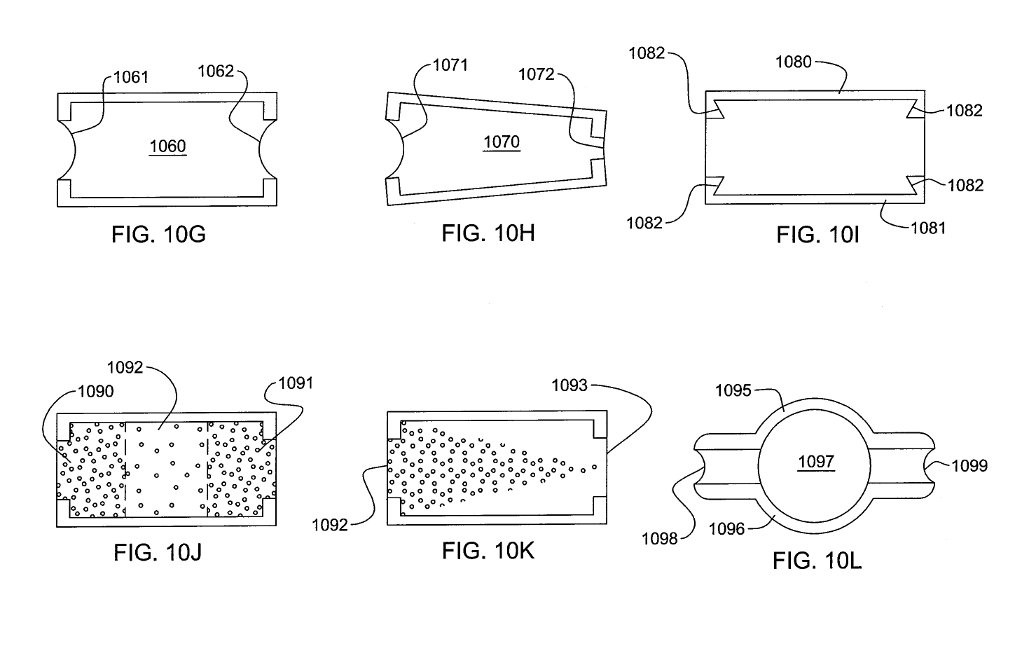

Further, although shown as similarly curved, exterior surfaces 521, 621 of the endplates in the embodiment of FIGS. 5-7A could be, in other embodiments, angled with respect to each other to accommodate a particular lordotic or kyphotic angle. As shown in FIG. 10H, the prosthetic device may be tapered, angled or wedge-shaped to achieve a desired lordotic or kyphotic angle. Such angles may be created by incorporating angled endplate assemblies and/or an angled non-articular, elongate core component (such as shown in FIG. 10H).

Referring to FIGS. 7 & 7A, intervertebral prosthetic device 500 may be assembled by frictionally fitting, for example, under slight compression, the non-articular, elongate flexible core component 610 within the respective first and second endplates 520, 620. Additionally, or alternatively, adhesive material may be applied to one or both of the elongate flexible core component and the interior surfaces of the endplates prior to assembly of the structure. Flat tether 550 may then be fed through aligned openings provided in the endplates and core component which are sized and configured to receive the tether. Alternatively, the flat tether could be fed through, for example, the second component, then the elongate flexible core, and finally the first component, as the components are sequentially assembled. Once fed through the elongate core to wrap around the first and second endplates, the tether is sealed to itself to define the tether loop illustrated in the figures. Any appropriate adhesive may be used to secure the flat tether to itself and form the loop. The assembled intervertebral prosthetic device 500 may then be implanted into an intervertebral disc space such that the exterior surfaces 521, 621 of the endplates 520, 620, as well as the first and second portions 551, 552 of tether 550 engage the vertebral endplates of the adjacent vertebral bodies.

In operation, the assembled intervertebral prosthetic device elastically deforms under compressive loads and elastically stretches in response to a force which may pull the endplates away from one another. The intervertebral prosthetic device may also deform or flex under flexion-extension or lateral bending motion. The flat tether advantageously flexes and constrains movement of the intervertebral prosthetic device responsive to one or more of these motions, while also reinforcing the device to provide enhanced operation of the prosthesis.

FIG. 7B illustrates an alternate embodiment of an intervertebral prosthetic device, generally denoted 700, in accordance with an aspect of the present invention. Intervertebral prosthetic device 700 is substantially identical to intervertebral prosthetic device 500 of FIGS. 5-7A and, unless otherwise stated, the description provided above in connection with device 500 of FIGS. 5-7A applies to device 700 of FIG. 7B as well.

As shown in FIG. 7B, intervertebral prosthetic device 700 includes a first component 520′ and a second component 620′ disposed in spaced relation via a non-articular, elongate flexible core component 610. In this embodiment, first component 520′ and second component 620′ each comprise a respective endplate assembly of the intervertebral prosthetic device. Each endplate assembly includes an endplate (such as described above in connection with the embodiment of FIGS. 5-7A), as well as a respective cover 710, 711. Covers 710, 711 can be manufactured of the same material described above in connection with the endplates of the intervertebral prosthetic device 500. As described further below, flat tether 550 again extends at least partially through first component 510′ and second component 620′ and forms a loop. This looped tether further extends through appropriate openings or channels formed in non-articular, elongate flexible core component 610. In operation, flat tether 550 again flexes yet constrains motion of the intervertebral prosthetic device when in operable position within an intervertebral disc space between adjacent vertebral bodies.

Each cover 710, 711 is configured (in this embodiment) to matably engage and substantially cover the respective endplate of the endplate assemblies 520′, 620′. Alternatively, covers 710, 711 could be configured to simply cover the first and second portions 551, 552 of flat tether 550 wrapping around the endplates. Further, frictional fitting of covers 710, 711 to their respective endplates may be employed or, alternatively, an adhesive material may be utilized between each cover and its respective endplate. Covers 710, 711 are shown to include exterior surfaces 720, 721, respectively, each of which have projecting therefrom a keel 730, 731. Each keel 730, 731 has multiple openings 740, 741 extending therethrough. Keels 730, 731 are sized and disposed to engage a respective vertebral endplate of the adjacent vertebral bodies when the prosthetic is implanted within the intervertebral disc space between the adjacent vertebral bodies, while openings 740, 741 promote bony in-growth and therefore fixation of the intervertebral prosthetic device to each adjacent vertebra. Additionally, exterior surfaces 720, 721 and keels 730, 731 may be roughened and/or coated with a biocompatible and osteoconductive material, or alternatively, an osteoinductive coating such as described above in connection with the embodiment of FIGS. 5-7A. These surfaces may be roughened by, for example, chemical etching, bead-blasting, sanding, grinding, serrating and/or diamond cutting.

As shown in FIG. 7B, each cover 710, 711 has an inner surface 722, 723 configured to accommodate the respective portions 551, 552 of flat tether 550 extending through the endplates of the endplate assemblies 520′, 620′. Alternatively, the endplates of the endplate assemblies 520′, 620′ could be configured with channels to accommodate the respective portions 551, 552 of flat tether 550 extending through the endplates. In either embodiment, one function of covers 710, 711 is to isolate flat tether 550 from contacting the respective vertebral endplates when the intervertebral prosthetic device is implanted within an intervertebral disc space of a patient.

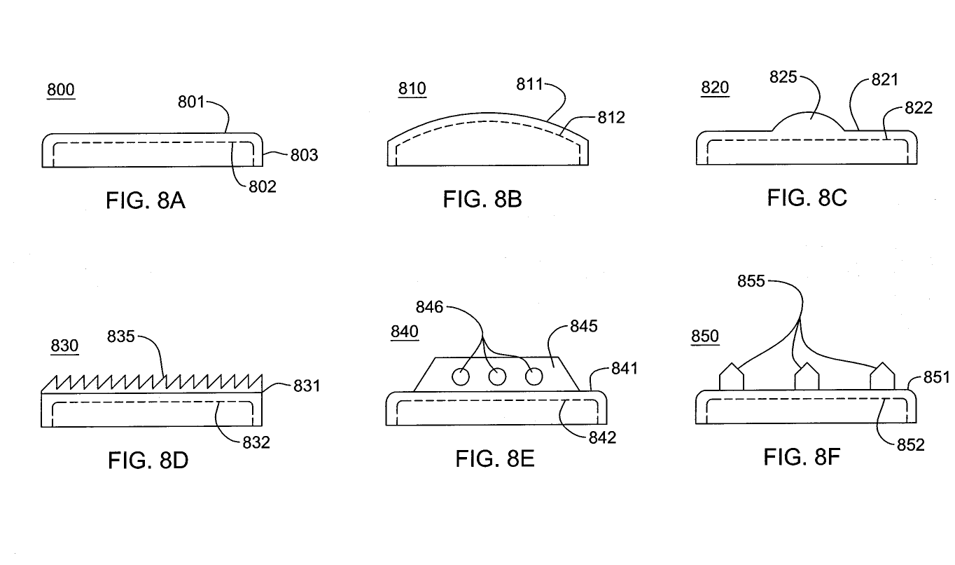

Numerous variations to the intervertebral prosthetic device embodiments depicted in FIGS. 5-7B are possible. By way of example, FIGS. 8A-8F depict various endplate or cover configurations usable with either intervertebral prosthetic device 500 (FIGS. 5-7A) or intervertebral prosthetic device 700 (FIG. 7B). In FIG. 8A, endplate 800 has a substantially flat exterior surface 801 and substantially flat interior surface 802, along with a circumferential lip 803 which facilitates securement of the non-articular, elongate flexible core component (not shown) to the endplate. In FIG. 8B, the exterior surface 811 of the endplate 810 is convex-shaped, and the interior surface is correspondingly concave-shaped 812, as in the device embodiment of FIGS. 5-7A. In this embodiment, the non-articular, elongate flexible core component would be configured such as core component 610 in the embodiments of FIGS. 5-7B. FIG. 8C depicts an endplate 820 configuration wherein exterior surface 821 includes a spherical segment 825 protruding therefrom. This spherical segment portion 825 may be sized and disposed to reside within a nuclear recess in an adjacent vertebral body when the intervertebral prosthetic device is implanted within an intervertebral disc space. Interior surface 822 of endplate 820 is shown to be flat in this example.

In FIG. 8D, endplate 830 includes a flat exterior surface 831 and flat interior surface 832, along with a serrated keel 835 projecting from exterior surface 831. In FIG. 8E, endplate 840 has a flat exterior surface 841, a flat interior surface 842, and a keel 845 projecting from exterior surface 841. In this embodiment, keel 845 includes multiple openings 846 disposed therein to facilitate bony in-growth when the endplate is in physical contact with a respective vertebral body. In FIG. 8F, endplate 850 is shown to include a flat exterior surface 851, a flat interior surface 852 and multiple fixation spikes 855 projecting from exterior surface 851.

Again, as noted above, these various embodiments of endplates 800, 810, 820, 830, 840 & 850 depicted in FIGS. 8A-8F are provided by way of example only. Although described herein as endplates, these structures could alternatively be examples of covers used to cover an endplate in an endplate assembly employed in an intervertebral prosthetic device embodiment such as depicted in FIG. 7B.

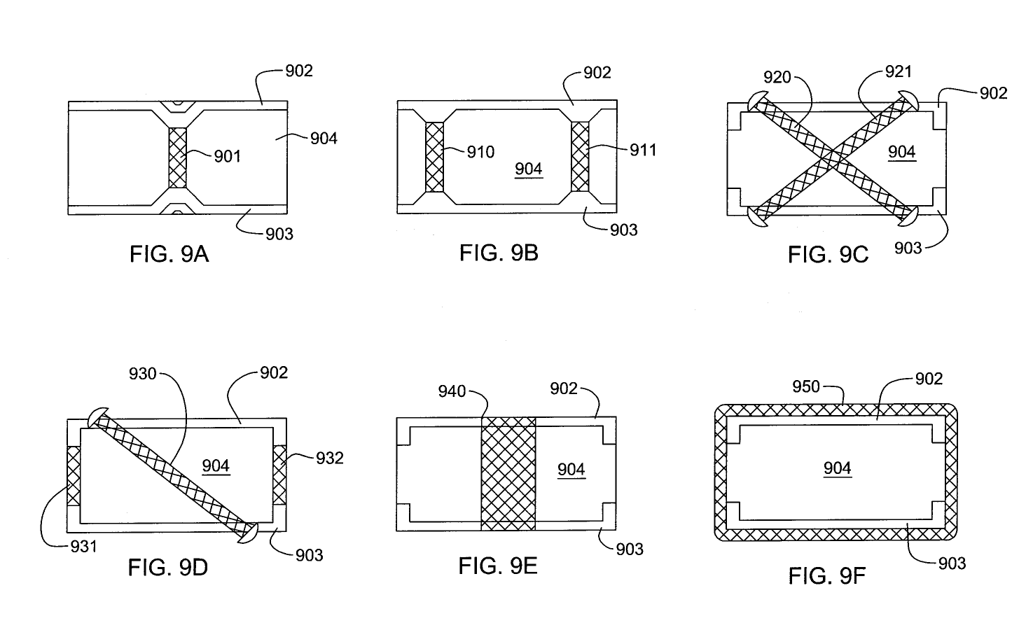

FIGS. 9A-9F depict various configurations for tethering a first component, second component and non-articular, elongate flexible core component. In each configuration, the flat tether is assumed to comprise an elastic tether having a higher modulus than the flexible core component. Further, with the exception of the embodiment of FIG. 9E, each flat tether is assumed to have a width extending into the figure, i.e., in a direction transverse to the longitudinal axis of the elongate intervertebral prosthetic device illustrated. In FIG. 9A, a single discrete tether 901 is shown connected between first component 902 and second component 903 through a centrally disposed channel within flexible core 904. Tether 901 can be connected to components 902, 903 using any appropriate mechanism, such as crimping, screws, adhesive, etc. In FIG. 9B, two discrete flat tethers are shown coupling first component 902, second component 903 and elongate flexible core 904. In FIG. 9C, two angled tethers 920, 921 are shown interconnecting first component 902, second component 903 and non-articular, elongate flexible core component 904. Angling of the one or more tethers as diagonal tethers may be beneficial depending upon the particular intervertebral disc space within which the intervertebral prosthetic device is to be inserted. For example, if the prosthetic device is configured with a particular lordotic angle, one or more diagonally disposed tethers may advantageously reinforce and constrain motion of the intervertebral prosthetic device. A variation on this concept is depicted in FIG. 9D wherein a single angled tether 930 interconnects first component 902, second component 903 and non-articular, elongate flexible core component 904. In this example, a first end tether 931 and a second end tether 932 are also disposed at a first end and a second end, respectively, of the core component to further reinforce and interconnect first component 902, second component 903 and non-articular, elongate flexible core component 904.

FIGS. 9E & 9F depict alternate embodiments of a looped tether such as depicted in the embodiments of FIGS. 5-7B. In these embodiments, however, the looped tether at least partially surrounds an exterior surface of first component 902, second component 903 and non-articular, elongate flexible core component 904. In the embodiment of FIG. 9E, the flat tether 940 encircles the components in a direction transverse to a longitudinal axis of the intervertebral prosthetic device. This embodiment may be advantageous in a lateral insertion approach. In FIG. 9F, the looped tether 950 at least partially longitudinally surrounds the first component 902, second component 903 and elongate flexible core 904, that is, encircles the components in a direction parallel to the longitudinal axis of the intervertebral prosthetic device.

From the above examples, it will be appreciated that the flat tether employed in an intervertebral prosthetic device such as presented herein can be of any one of various sizes, configurations, angles, etc. However, in each embodiment, the tether is a flat, flexible tether which flexes, yet constrains motion of the intervertebral prosthetic device.

FIGS. 10A-10L depict various elongate flexible core configurations for an intervertebral prosthetic device such as described herein. Although not shown in FIGS. 10A-10L, it should be understood from the above description that in each embodiment depicted, one or more flat tethers would also be employed to couple the first component, second component and non-articular, elongate flexible core component. In FIGS. 10A-10K, the first and second components are substantially flat components, while in FIG. 10L, the first and second components include spherical-shaped protrusions sized to accommodate a spherical-shaped center region of the non-articular, elongate flexible core component.

In the embodiment of FIG. 10A, the core component includes a first end region 1000, a second end region 1001 and a center region 1002, which (as shown) separates the first and second end regions 1000, 1001. The center region in this example is a partially spherical-shaped center region. End regions 1000, 1001 are assumed to comprise a lower modulus material than center region 1002. Thus, in this embodiment, end regions 1000, 1001 have greater elasticity than center region 1002. In the embodiment of FIG. 10B, the opposite structure is depicted, wherein end regions 1000′, 1001′ have a higher modulus than center region 1002′. In either embodiment, the regions of higher modulus assist in reinforcing the regions of lower modulus.

In the embodiment of FIG. 10C, a first region 1010 of the elongate flexible core has a different modulus than a second region 1020 of the elongate flexible core. For example, first region 1010 may comprise an anterior region and have a lower modulus than second region 1020, and thus be designed for posterior insertion between a particular set of vertebrae.

In FIGS. 10D, 10E & 10F, elasticity of the elongate flexible core at least partially progressively varies from a first end to a second end thereof. For example, in FIG. 10D, multiple regions 1030 are disposed within elongate flexible core 1031. These multiple regions have a common geometric shape, and reduce in size from the first end to the second end of the non-articular, elongate flexible core component 1031. Further, regions 1030 are assumed to have a different modulus than the balance of the core component. The embodiment of FIG. 10E is identical to that of FIG. 10D, except that the multiple regions 1040 are shown to be rectangular in shape (as opposed to spherical-shaped regions in the embodiment of FIG. 10D). Again, regions 1040 are assumed to have a different modulus than the balance of the core component 1041. Regions 1030, 1040 may be any material designed to exhibit a different degree of rigidity than the balance of the core component. This material may be employed to control, adjust, or modify the hardness, stiffness, flexibility, or compliance of the core component. These regions may be of any size, shape or material to permit variation in the rigidity of the core component. However, in the embodiment of FIGS. 10D & 10E, there is a partial progressive variation between the first end and the second end of the core component. The regions 1030, 1040 may be discrete bodies within the core component or have a gradient quality which allows the regions to blend into the balance of the core component between the first end to the second end. As a further alternative, regions 1030, 1040 could comprise voids within the non-articular, elongate flexible core component.

The regions 1030, 1040 may be formed from materials different than the core component, including any of the materials described above for the endplates or the core component. The materials may be stiffer or more pliable than the material of the core component. Further, if the regions 1030, 1040 are voids, then in certain embodiments, one or more of these voids may function as reservoirs for therapeutic agents such as analgesics, anti-inflammatory substances, growth factors, antibiotics, steroids, pain medications, or combinations of agents. Growth factors may comprise any member of the families of transforming growth factor beta (TGH-beta), bone morphogenic proteins (BMPs), recombinant human bone morphogenic proteins (rh BMPs), insulin-like growth factors, platelet-derived growth factors, fibroblast growth factors, or any other growth factors that help promote tissue repair of surrounding tissues.

In FIG. 10F, the elongate flexible core is constructed to have a progressively changing modulus from one end to the other end. For example, the modulus of the elongate flexible core 1050 could progressively increase from a first end 1051 to a second end 1052 thereof. This can be achieved by a number of techniques. For example, porosity of elongate flexible core 1050 could vary from first end 1051 to second end 1052, as described below in connection with FIG. 10K. Alternatively, controlled reactive injection molding could be employed to inject different levels of cross-linking material into the elongate flexible core during formation of the core. That is, a progressively higher amount of cross-linking could be employed from the first end 1050 to the second end 1052 of the elongate flexible core during fabrication thereof, resulting in a progressively changing modulus from a lower modulus end (e.g., first end 1051) to a higher modulus end (e.g., second end 1052). As one example, less cross-linking may be employed at an anterior end of the intervertebral prosthetic device, and more cross-linking at a posterior end thereof.

As a further alternative approach, two different materials may be mixed to form a composite elongate flexible core, with one material having a higher modulus than the other material. In this approach, the concentrations of the first and second materials can be progressively varied as the materials are injected into a mold of the elongate flexible core, with (for example) the higher modulus material having a higher concentration near the posterior end of the intervertebral prosthetic device, and the lower modulus material having a higher concentration near the anterior end thereof.

In FIG. 10G, the intervertebral prosthetic device includes a non-articular, elongate flexible core component 1060 having a first end 1061 and a second end 1062, wherein first and second ends 1061, 1062 are each configured as a concave surface to reduce stiffness and enhance motion of the intervertebral prosthetic device at the ends thereof. In FIG. 10H, the non-articular, elongate flexible core component 1070 is tapered, angled or wedge-shaped from a first end 1071 to a second end 1072 thereof. This tapering, angling or wedge-shaped design of core component 1070 may be employed to achieve a desired lordotic or kyphotic angle. Alternatively, the prosthesis could be angled by incorporating angled endplates, or by incorporating flat endplates with a core component having only one angled side. The prosthesis of FIG. 10H is angled by incorporating flat endplates with a core component having two angled sides. Further, in the example of FIG. 10H, first end 1071 of elongate flexible core 1070 further comprises a concave surface to again reduce stiffness and enhance motion at the first end of the intervertebral prosthetic device.

In FIG. 10I, an alternative intervertebral prosthetic device embodiment is depicted wherein the first component 1080 and second component 1081 each include dovetail-shaped protrusions or structures 1082 extending therefrom disposed at the first and second ends of the core component. These dovetail-shaped protrusions 1082 facilitate fixedly securing the core component to first component 1080 and second component 1081.

In FIGS. 10J & 10K, variations in porosity are employed to achieve regions of different elasticity within the elongate flexible core. In FIG. 10J, a first end region 1090 and a second end region 1091 are shown disposed at a first end and a second end of the core component. Regions 1090, 1091 are separated by a center region 1092 of lower porosity, and thus higher modulus than the end regions. This embodiment is geometrically similar to the embodiment depicted in FIG. 10A, except that the center region is rectangular-shaped rather than partially spherically-shaped, as in the embodiment of FIG. 10A. In FIG. 10K, porosity of the elongate flexible core at least partially decreases from a first end 1092 to a second end 1093 thereof. Thus, the modulus at least partially progressively increases from first end 1092 to second end 1093 of this core component embodiment.

In FIG. 10L, a further variation is depicted wherein the first component 1095 and second component 1096 are fabricated with a spherical (or cylindrical) segment protrusion extending therefrom sized and disposed to accommodate a spherically-shaped (or cylindrically-shaped) center region 1097 with the non-articular, elongate flexible core component. In this embodiment, center region 1097 may be a higher modulus material for enhanced load bearing capabilities of the intervertebral prosthetic device. Further, in this embodiment, the flexible core component is shown to be concave at a first end 1098 and at a second end 1099 thereof to further reduce stiffness and enhance motion of the device in these regions.

As noted above, FIGS. 11-27 further illustrate various device configurations and depict bilateral and unilateral posterior implant processes which could be employed, in accordance with certain aspects of the present invention.

In FIG. 11, two identical, rectangular-shaped intervertebral prosthetic devices 1100 are shown inserted posteriorally into an intervertebral disc space through appropriate bilarteral incisions in the patient's back and appropriate openings on lateral side 1101 and lateral side 1102 of the disc space, providing access to the intervertebral disc space.

FIG. 12 depicts an alternate, unilateral embodiment wherein a single intervertebral prosthetic device 1200, such as described above in connection with FIGS. 5-10L is inserted into an intervertebral disc space via a posterior opening on one lateral side 1202 of the intervertebral disc space.

FIGS. 13-18 depict various alternate configurations for an intervertebral prosthetic device, in accordance with an aspect of the present invention. In each embodiment, it is assumed that the intervertebral prosthetic device is constructed as described above in connection with the embodiments of FIGS. 5-10L, and is being posteriorally inserted through respective lateral side openings into the illustrated intervertebral disc space. In the embodiment of FIG. 13, two kidney-shaped intervertebral prosthetic devices 1300 are inserted. Again, in one embodiment, kidney-shaped intervertebral prosthetic device 1300 may be fabricated with kidney-shaped superior and inferior endplates, and a kidney-shaped core component. Similarly, FIG. 14 illustrates insertion of two semi-circular intervertebral prosthetic devices 1400, FIG. 15 illustrates insertion of two triangular-shaped intervertebral prosthetic devices 1500, FIG. 16 illustrates insertion of two trapezoidal-shaped intervertebral prosthetic devices 1600, FIG. 17 illustrates insertion of two mating rectangular-shaped intervertebral prosthetic devices 1700, and FIG. 18 illustrates insertion of two mating semi-toroidal-shaped intervertebral prosthetic devices 1800. FIGS. 17 & 18 illustrate that the intervertebral prosthetic devices being inserted bilaterally could be designed to engage or couple in situ once positioned within the intervertebral disc space. Similarly, although not shown, kidney-shaped intervertebral prosthetic devices 1300 could optionally be placed in mating engagement, for example, at their anterior ends, once implanted into the intervertebral disc space. Semi-circular intervertebral prosthetic devices 1400 and triangular-shaped intervertebral prosthetic devices 1500 could also readily be placed in engaging relation along their opposing surfaces once implanted into the intervertebral disc space. Trapezoidal-shaped intervertebral prosthetic devices 1600 may be employed to more advantageously match a particular intervertebral disc space within which the devices are to be implanted.

In the embodiments of FIGS. 19 & 20, two similarly-shaped but differently sized intervertebral prosthetic devices are bilaterally, posteriorally inserted. In the process of FIG. 19, a first intervertebral prosthetic device 1900 may be inserted through one posterior opening (not shown) and rotated as shown, followed by insertion of the second intervertebral prosthetic device 1901 through a second posterior opening (not shown), followed by rotation thereof into the position illustrated. FIG. 20 illustrates a similar intervertebral prosthetic device insertion process to that of FIG. 19, with the exception that the differently sized intervertebral prosthetic devices 2000, 2001 are kidney-shaped devices in the process of FIG. 20.

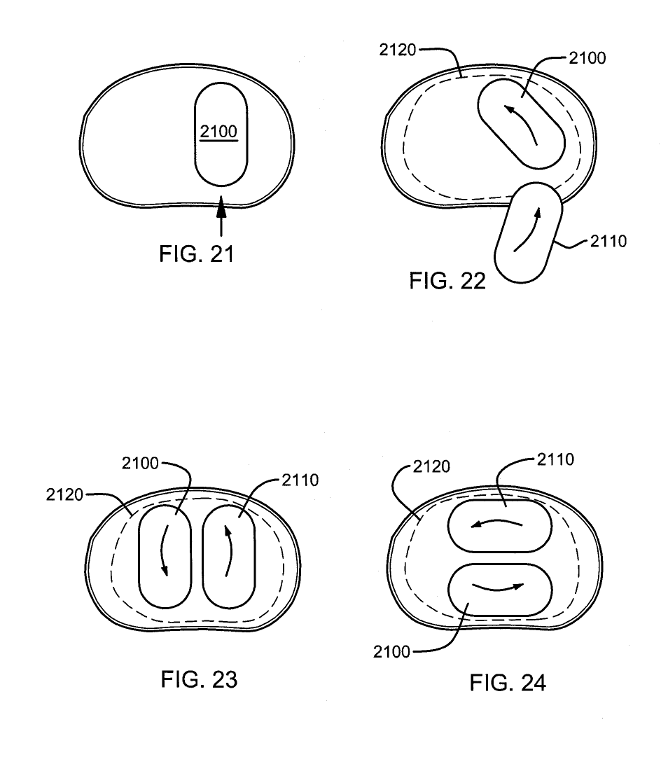

FIGS. 21-24 depict a further implant process wherein a first intervertebral prosthetic device 2100 is inserted through a unilateral, posterior opening of the intervertebral disc space (FIG. 21), followed by a second intervertebral prosthetic device 2110 (FIG. 22). As the second intervertebral prosthetic device 2110 is inserted it may engage and advance first intervertebral prosthetic device 2100, pushing it from its original position. First intervertebral prosthetic device 2100 may travel along an arcuate guide path or a recessed surface 2120 created along the annulus of the intervertebral disc space. In an alternate embodiment, the bone of the adjacent endplate may be prepared to guide the intervertebral prosthetic device 2100 along the guide path 2120.

As shown in FIG. 23, intervertebral prosthetic device 2110 may be fully inserted into the intervertebral disc space. As this prosthetic device becomes inserted, the first intervertebral prosthetic device 2100 may continue along the arcuate path 2120 until coming to rest on the opposite lateral side of the intervertebral disc space. As shown in FIG. 24, to mitigate the risk of intervertebral prosthetic device 2110 becoming expulsed through the posterior opening, both of the prosthetic devices may continue to be positioned along the arcuate path until the intervertebral prosthetic devices 2100, 2110 extend across the intervertebral disc space and into both lateral sides of the disc space. Alternatively, it is to be understood that the prosthetic devices 2100, 2110 may remain in the position illustrated in FIG. 23 with other structures or techniques used to prevent expulsion of the components.

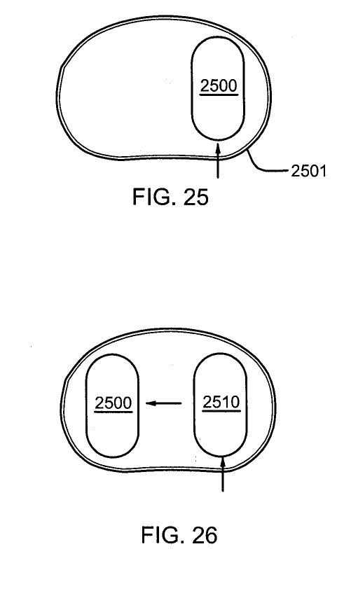

FIGS. 25 & 26 depict an alternative process for implanting two intervertebral prosthetic devices such as described above in connection with FIGS. 5-10L. A posterior unilateral opening is first created on one lateral side of the intervertebral prosthetic device. Through this opening, instrumentation may be inserted to evacuate the remaining disc tissue. Instrumentation may also be inserted to mill or to otherwise dislocate bone to fashion a path or recess in one or both of the endplates adjacent to the intervertebral disc space. It is understood that in some embodiments, no bone removal may be needed.

As shown in FIG. 25, a first intervertebral prosthetic device 2500 is inserted through a posterior lateral 2501 opening into the intervertebral disc space. As shown in FIG. 26, intervertebral prosthetic device 2500 may be displaced from lateral side 2501 and shuttled to the opposite lateral side using, for example, an instrument or alternatively, a second intervertebral prosthetic device 2510 as a pushing tool as it is being inserted into the intervertebral disc space.

The use of a posterior approach such as described above in connection with FIGS. 21-26 may offer the surgeon a technique similar to fusion with which the surgeon may already be familiar. The posterior approach may allow herniations impinging on a nerve root to be more easily decompressed. Further, later revision surgeries may be more easily managed as compared to anteriorally placed devices.

Alternatively, as noted above, a lateral approach to the intervertebral disc space could be employed to unilaterally or bilaterally insert one or two intervertebral prosthetic devices such as described above in connection with FIGS. 5-10L. Depending upon whether the device is to be posteriorally or laterally inserted, various characteristics thereof may be chosen. For example, in a lateral insertion approach, it may be beneficial to employ a flat looped tether transverse to the longitudinal axis of the device, as illustrated in FIG. 9E.

Although certain preferred embodiments have been depicted and described in detail herein, it will be apparent to those skilled in the relevant art that various modifications, additions and substitutions can be made without departing from the concepts disclosed and therefore these are to be considered to be within the scope of the following claims. For example, although the devices and methods of the present invention are particularly applicable to the lumbar region of the spine, it should nevertheless be understood that the present invention is also applicable to other portions of the spine, including the cervical or thoracic regions of the spine.

What is claimed is:

1. An intervertebral prosthetic device comprising:
a first component adapted to engage a first vertebral body;
a second component adapted to engage a second vertebral body;
a non-articular, elongate flexible core component interposed between and fixedly secured to the first and second components, and biasing the first and second components in spaced relation; and
at least one flat tether connecting the first component and the second component to further bind together the first component, the non-articular, elongate flexible core component and the second component, and to constrain motion of the intervertebral prosthetic device when in an operable position within an intervertebral disc space between the first and second vertebral bodies, and subject to at least one of a flexion or extension force, a lateral bending force or an axial rotation force.

2. The intervertebral prosthetic device of claim 1, wherein the at least one flat tether is a looped tether wrapping around at least a portion of the first component and the second component.

3. The intervertebral prosthetic device of claim 2, wherein the looped tether loops through the non-articular, elongate flexible core component.

4. The intervertebral prosthetic device of claim 3, wherein a first portion of the looped tether engages the first vertebral body and a second portion of the looped tether engages the second vertebral body when the intervertebral prosthetic device is in operable position between the first and second vertebral bodies, and wherein the first and second portions of the looped tether are coated with a biological factor to facilitate bony fixation of the intervertebral prosthetic device to the first and second vertebral bodies.

5. The intervertebral prosthetic device of claim 4, wherein the first component and the second component each have a length that extends upon cortical bone of opposing sides of an apophyseal ring of the corresponding vertebral body of the first and second vertebral bodies, and a width that is smaller than the length, and wherein the non-articular, elongate flexible core has a length and width at most equal to a length and width, respectively, of the first component and the second component.

6. The intervertebral prosthetic device of claim 2, wherein the looped tether further wraps around at least a portion of the non-articular, elongate flexible core component, and wherein the looped tether is a flexible, braided, textile-based structure having a width W and a thickness T, wherein width W>thickness T.

7. The intervertebral prosthetic device of claim 6, wherein the first component and the second component each have a length that extends upon cortical bone of opposing sides of an apophyseal ring of the corresponding vertebral body of the first and second vertebral bodies, and a width that is smaller than the length, and wherein the non-articular, elongate flexible core component has a length and width at most equal to a length and width, respectively, of the first component and the second component.

8. The intervertebral prosthetic device of claim 7, wherein the looped tether extends around the at least a portion of the first component, second component and non-articular, elongate flexible core component in a direction parallel to a length dimension of the intervertebral prosthetic device.

9. The intervertebral prosthetic device of claim 7, wherein the looped tether extends around the at least a portion of the first component, second component and non-articular, elongate flexible core component in a direction transverse to a length dimension of the intervertebral prosthetic device.

10. The intervertebral prosthetic device of claim 1, wherein each at least one flat tether is a discrete tether extending through the non-articular, elongate flexible core component, each discrete tether being secured at a first end to the first component and at a second end to the second component.

11. The intervertebral prosthetic device of claim 10, wherein each discrete tether is a flexible, braided, textile-based structure.

12. The intervertebral prosthetic device of claim 10, wherein the at least one discrete tether comprises a centrally disposed flat tether extending between the first component and the second component through the non-articular, elongate flexible core component.

13. The intervertebral prosthetic device of claim 1, wherein the at least one flat tether comprises an angled tether extending at a diagonal angle through the non-articular, elongate flexible core component from the first component to the second component, the angled tether being disposed to resist a shear load applied to the intervertebral prosthetic device.

14. The intervertebral prosthetic device of claim 10, further comprising multiple discrete flat tethers connecting the first component and the second component to further bind together the first component, the non-articular, elongate flexible core component, and the second component, wherein the multiple discrete flat tethers each comprises a flexible, braided, textile-based structure.

15. The intervertebral prosthetic device of claim 14, wherein the multiple discrete flat tethers comprise a first flat tether and a second flat tether, the first flat tether and the flat second tether each extending through the non-articular, elongate flexible core and connecting the first component and the second component adjacent to a respective end of the intervertebral prosthetic device.

16. The intervertebral prosthetic device of claim 14, wherein the multiple discrete flat tethers comprise a first flat tether disposed at a first end of the non-articular, elongate flexible core component and a second flat tether disposed at a second end of the non-articular, elongate flexible core component, the first and second flat tethers each connecting the first component and the second component external to the non-articular, elongate flexible core component, and wherein the multiple discrete flat tethers further comprise a third diagonal tether extending through the non-articular, elongate flexible core component, and connecting the first component and the second component.

17. The intervertebral prosthetic device of claim 14, wherein the multiple discrete flat tethers comprise a first diagonal flat tether and a second diagonal flat tether extending through the non-articular, elongate flexible core at intersecting angles and connecting the first component and the second component.

18. The intervertebral prosthetic device of claim 1, wherein the at least one flat tether is at least one flexible, braided, textile-based structure having a width W and a thickness T, wherein width W>thickness T.

19. The intervertebral prosthetic device of claim 18, wherein the first component comprises a first endplate assembly and the second component comprises a second endplate assembly, the first endplate assembly including a first cover configured to engage the first vertebral body and at least partially cover a first endplate, and the second endplate assembly including a second cover configured to engage the second vertebral body and at least partially cover a second endplate, and wherein the first endplate assembly and the second endplate assembly receive respective portions of the at least one flat tether and isolate the respective portions of the at least one flat tether from contacting the first vertebral body and second vertebral body when the intervertebral prosthetic device is in operable position within the intervertebral disc space between the first and second vertebral bodies.

20. The intervertebral prosthetic device of claim 19, wherein the first cover and the second cover each further comprise one of an osteoconductive or osteoinductive coating on an exposed surface thereof in physical contact with a respective vertebral body of the first and second vertebral bodies when the intervertebral prosthetic device is in operable position between the first and second vertebral bodies.

21. The intervertebral prosthetic device of claim 19, wherein the first cover and the second cover each comprise at least one of an exposed convex surface, an exposed surface with a spherical segment protruding therefrom, an exposed surface with a keel extending therefrom comprising at least one of a serrated edge or multiple holes therein for facilitating bony in-growth, or an exposed surface with at least one spike protruding therefrom.

22. An intervertebral prosthetic device comprising:
a first component adapted to engage a first vertebral body;
a second component adapted to engage a second vertebral body;
a non-articular, elongate flexible core component interposed between and secured to the first and second components, the non-articular, elongate flexible core component biasing the first and second components in spaced relation, and comprising regions of different elasticity; and
at least one flat tether coupled to connect the first component and the second component to further bind together the first component, non-articular, elongate flexible core component and second component, and to constrain motion of the intervertebral prosthetic device when in an operable position between the first and second vertebral bodies, and subject to at least one of a flexion or extension force, a lateral being force of an axial rotation force.

23. The intervertebral prosthetic device of claim 22, wherein the first component comprises a first endplate assembly and the second component comprises a second endplate assembly, the first endplate assembly including a first cover configured to engage the first vertebral body and at least partially cover a first endplate, and the second endplate assembly including a second cover configured to engage the second vertebral body and at least partially cover a second endplate, and wherein the first endplate assembly and second endplate assembly receive respective portions of the at least one flat tether and isolate the respective portions of the at least one flat tether from contacting the first vertebral body and the second vertebral body.

24. The intervertebral prosthetic device of claim 23, wherein the first cover includes at least one fixation feature projecting from an exterior surface thereof, and the second cover includes at least one fixation feature projecting from an exterior surface thereof, each fixation feature comprising at least one of a spike, a roughened keel, a serrated keel, a keel with multiple openings, a diamond-cut surface or other roughened surface.

25. The intervertebral prosthetic device of claim 23, wherein the first cover is configured to mate with the first endplate and the second cover is configured to mate with the second endplate, and wherein at least one of the first cover or the first endplate, and at least one of the second cover or the second endplate includes a groove for receiving the respective portion of the at least one flat tether.

26. The intervertebral prosthetic device of claim 23, wherein the first component and the second component each have a length that extends upon cortical bone of opposing sides of an apophyseal ring of the corresponding vertebral body of the first and second vertebral bodies, and a width that is smaller than the length, and wherein the non-articular, elongate flexible core component has a length and width at most equal to a length and width, respectively, of the first component and the second component.

27. The intervertebral prosthetic device of claim 22, wherein the first component, the non-articular, elongate flexible core component and the second component are configured to mechanically interlock and thereby secure the non-articular, elongate flexible core component relative to the first and second components.

28. The intervertebral prosthetic device of claim 27, wherein the mechanical interlocking comprises dovetail-shaped protrusions extending from the first component and from the second component at a first end and at a second end of the non-articular, elongate flexible core component, the dovetail-shaped protrusions fixedly securing the non-articular, elongate flexible core component relative to the first component and the second component.

29. The intervertebral prosthetic device of claim 22, wherein a first portion of the at least one flat tether engages the first vertebral body and a second portion of the at least one flat tether engages the second vertebral body when the intervertebral prosthetic device is in an operable position between the first and second vertebral bodies, and wherein the first and second portions of the at least one flat tether are coated with a biological factor to facilitate bony fixation of the intervertebral prosthetic device to the first and second vertebral bodies when implanted between the first and second vertebral bodies.

30. The intervertebral prosthetic device of claim 22, wherein the non-articular, elongate flexible core component comprises a first end and a second end, and the regions of different elasticity comprise first and second end regions of the non-articular, elongate flexible core component separated by a center region, and wherein the non-articular, elongate flexible core component has one of a lower modulus in the first and second end regions than in the center region or a higher modulus in the first and second end regions than in the center region.

31. The intervertebral prosthetic device of claim 30, wherein the center region is an at least partially spherical-shaped region of the non-articular, elongate flexible core component.

32. The intervertebral prosthetic device of claim 30, wherein the first and second end regions have a greater porosity than the center region.

33. The intervertebral prosthetic device of claim 30, wherein the center region comprises a spherical-shaped load-bearing region of higher modulus than the first and second end regions.

34. The intervertebral prosthetic device of claim 33, wherein an end surface of at least one of the first end or the second end of the non-articular, elongate flexible core component is concave to reduce stiffness and to enhance motion of the intervertebral prosthetic device at the at least one first or second end.

35. The intervertebral prosthetic device of claim 22, wherein the non-articular, elongate flexible core component comprises a first end and a second end, and wherein elasticity of the non-articular, elongate flexible core component at least partially progressively varies between the first end and the second end thereof.

36. The intervertebral prosthetic device of claim 35, wherein porosity of the non-articular, elongate flexible core component at least partially progressively varies between the first end and the second end thereof.

37. The intervertebral prosthetic device of claim 35, wherein the non-articular, elongate flexible core component further comprises multiple regions of different modulus disposed within the non-articular, elongate flexible core component, wherein the multiple regions of different modulus reduce in size between the first end and the second end of the non-articular, elongate flexible core component.

38. The intervertebral prosthetic device of claim 37, wherein the multiple regions of different modulus comprise multiple voids formed in the non-articular, elongate flexible core component, the multiple voids in the non-articular, elongate flexible core component reducing in size between the first end and the second end thereof.

39. The intervertebral prosthetic device of claim 37, wherein the multiple regions of different modulus are multiple regions of a common geometric shape, but reducing geometric size between the first end and the second end.

40. The intervertebral prosthetic device of claim 22, wherein the non-articular, elongate flexible core component comprises a first end and a second end, and the regions of different elasticity comprise a first region extending to the first end and a second region extending to the second end, wherein elasticity of the first region is different from elasticity of the second region.

41. The intervertebral prosthetic device of claim 22, wherein the non-articular, elongate flexible core component comprises a first end and a second end, and wherein an end surface of at least one of the first end or the second end is concave to reduce stiffness and to enhance motion of the intervertebral prosthetic device at the at least one first or second end.

42. The intervertebral prosthetic device of claim 22, wherein height of the non-articular, elongate flexible core component varies from the first end to the second end, the first end of the non-articular, elongate flexible core component being at an anterior end of the intervertebral prosthetic device and the second end being at a posterior end of the intervertebral prosthetic device, and wherein the non-articular, elongate flexible core component is tapered from the first end to the second end to facilitate restoring lordosis when inserted into an intervertebral disc space between the first and second vertebral bodies, and wherein an end surface at the first end of the non-articular, elongate flexible core component is concave.

43. The intervertebral prosthetic device of claim 22, wherein the non-articular, elongate flexible core component is fabricated of an elastomer material, the elastomer material comprising at least one channel sized to receive the at least one flat tether, and wherein the at least one flat tether is a flexible, braided, textile-based structure having a width W and a thickness T, wherein width W>thickness T.

44. An intervertebral prosthetic device comprising:
a first component adapted to engage a first vertebral body;
a second component adapted to engage a second vertebral body;
a non-articular, elongate flexible core component interposed between and secured to the first and second components to bias the first and second components in spaced relation;
at least one tether connecting the first component and the second component to further bind together the first component, the non-articular, elongate flexible core component and the second component, and to constrain motion of the intervertebral prosthetic device when inserted in an operable position within an intervertebral disc space between the first and second vertebral bodies, and subject to at least one of a flexion or extension force, a lateral being force or an axial rotation force; and
wherein the first component and the second component each have a length that extends upon cortical bone of opposing sides of an apophyseal ring of the corresponding vertebral body of the first and second vertebral bodies, and a width that is smaller than the length, and wherein the non-articular, elongate flexible core component has a length and width at most equal to a length and width, respectively, of the first component and the second component.

45. The intervertebral prosthetic device of claim 44, wherein the non-articular, elongate flexible core component is fixedly secured to the first and second components to prevent articular motion between the first component and the non-articular, elongate flexible core component and to prevent articular motion between the second component and the non-articular, elongate flexible core component.

46. The intervertebral prosthetic device of claim 45, wherein the at least one tether is a flat looped tether wrapping around at least a portion of the first component and the second component.

47. The intervertebral prosthetic device of claim 45, wherein the at least one tether is a discrete flat tether extending through the non-articular, elongate flexible core component, the discrete flat tether being secured at a first end to the first component and at a second end to the second component.

48. The intervertebral prosthetic device of claim 44, wherein the first component comprises a first exposed surface in contact with the first vertebral body and the second component comprises a second exposed surface in contact with the second vertebral body when the intervertebral prosthetic device is in operable position within the intervertebral disc space between the first and second vertebral bodies, the first exposed surface and the second exposed surface each being at least one of an exposed convex surface, an exposed surface with a spherical segment protruding therefrom, an exposed surface with a keel extending therefrom comprising at least one of a roughened surface, a serrated edge or multiple holes extending therethrough for facilitating bony in-growth, or an exposed surface with at least one spike protruding therefrom.

49. The intervertebral prosthetic device of claim 44, wherein the first component, the non-articular, elongate flexible core component and the second component mechanically interlock via dovetail-shaped protrusions extending from the first component and the second component.

50. The intervertebral prosthetic device of claim 44, wherein a first portion of the at least one tether engages the first vertebral body and a second portion of the at least one tether engages the second vertebral body when the intervertebral prosthetic device is inserted in operable position between the first and second vertebral bodies, and wherein the first and second portions of the at least one tether are coated with a biological factor to facilitate bony fixation of the intervertebral prosthetic device to the first and second vertebral bodies when implanted between the first and second vertebral bodies.

51. The intervertebral prosthetic device of claim 44, wherein the first component comprises a first endplate assembly and the second component comprises a second endplate assembly, the first endplate assembly including a first cover configured to engage the first vertebral body and at least partially cover a first endplate, and the second endplate assembly including a second cover configured to engage the second vertebral body and at least partially cover a second endplate, and wherein the first endplate assembly and the second endplate assembly receive respective portions of the at least one tether and isolate respective portions of the at least one tether from contacting the first vertebral body and the second vertebral body when the intervertebral prosthetic device is in operable position within the intervertebral disc space between the first and second vertebral bodies.

52. The intervertebral prosthetic device of claim 44, wherein elasticity of the non-articular, elongate flexible core component at least partially varies between a first end and a second end thereof.

53. The intervertebral prosthetic device of claim 52, wherein elasticity of the non-articular, elongate flexible core component at least partially progressively varies between the first end and the second end thereof.

54. The intervertebral prosthetic device of claim 44, wherein the intervertebral prosthetic device has a width less than 15 mm, a length in the range of 18-30 mm, and a height in the range of 8-18 mm.

55. The intervertebral prosthetic device of claim 44, wherein the first component, the second component and the elongate, non-articular flexible core component each have one of a rectangular shape, a kidney shape, a semi-circular shape, a triangular shape, a trapezoid shape, or a semi-toroidal shape.

56. A method for implanting an intervertebral prosthetic device into an intervertebral disc space, the method comprising:
obtaining an intervertebral prosthetic device comprising:
a first component adapted to engage a first vertebral body;
a second component adapted to engage a second vertebral body;
a non-articular, elongate flexible core component interposed between and fixedly secured to the first and second components, the non-articular, elongate flexible core component biasing the first and second components in spaced relation; and
at least one flat tether connecting the first component and the second component to further bind together the first component, the non-articular, elongate flexible core component and the second component, and to constrain motion of the intervertebral prosthetic device when in an operable position within an intervertebral disc space between the first and second vertebral bodies, and subject to at least one of a flexion or extension force, a lateral bending force or an axial rotation force;
surgically accessing an intervertebral disc space through an opening on a lateral side of the intervertebral disc space; and
inserting the intervertebral prosthetic device into the intervertebral disc space through the opening on the lateral side of the intervertebral disc space and positioning the first component in engagement with the first vertebral body and the second component in engagement with the second vertebral body.

57. The method of claim 56, further comprising obtaining two intervertebral prosthetic devices and wherein the method further comprises inserting the two intervertebral prosthetic devices into the intervertebral disc space.

58. The method of claim 57, wherein the inserting further comprises bilaterally, posteriorally inserting the two intervertebral prosthetic devices into the intervertebral disc space through openings on first and second lateral sides of the intervertebral disc space.

59. The method of claim 57, wherein the inserting further comprises sequentially inserting the two intervertebral prosthetic devices through the opening on the lateral side of the intervertebral disc space.

60. The method of claim 56, wherein the opening on the lateral side of the intervertebral disc space is a posterior, lateral opening to the intervertebral disc space.

61. The method of claim 56, further comprising obtaining two intervertebral prosthetic devices, and wherein the method further comprises inserting the two intervertebral prosthetic devices into the intervertebral disc space, and matably engaging in operative position the two intervertebral prosthetic devices in situ within the intervertebral disc space.

62. The method of claim 61, wherein the matably engaging comprises matably engaging opposing surfaces of the two intervertebral prosthetic devices in operative position within the intervertebral disc space.
